(19) United States
(12) Patent Application Publication
(10) Pub. No.: US 2012/0143141 A1
(43) Pub. Date: Jun. 7, 2012

(54) CONFORMAL CANNULA DEVICE AND RELATED METHODS

(76) Inventors: Josiah E. Verkaik, Lompoc, CA (US); James F. Antaki, Pittsburgh, PA (US); John Alexander Martin, Park City, UT (US)

(21) Appl. No.: 13/197,605

(22) Filed: Aug. 3, 2011

Related U.S. Application Data
(60) Provisional application No. 61/370,360, filed on Aug. 3, 2010.

(51) Int. Cl. A61M 25/04

(52) U.S. Cl. 604/175; 604/174

(57) ABSTRACT

Cannula assemblies and related methods are provided. In accordance with one embodiment, a cannula assembly includes a tubular structure coupled with a flange assembly. The flange assembly includes a plurality of wireform loops disposed in a circumferential, woven pattern about an end of the tubular structure. The flange assembly is configured to exhibit a first, collapsed state wherein the plurality of wireform loops extend substantially axially from the tubular structure, and a second, expanded state wherein the plurality of wireform loops extend in a direction having a substantial radial component relative to the tubular structure. In another embodiment, a cannula assembly includes a conformal flange coupled with a tubular structure, wherein the tubular structure extends both distally and proximally of the flange.

### CROSS-REFERENCE TO RELATED APPLICATIONS

The present application claims priority to U.S. Provisional Application No. 61/370,360, filed on Aug. 3, 2010, entitled CONFORMAL CANNULA DEVICE, the disclosure of which is incorporated by reference herein in its entirety.

### TECHNICAL FIELD

Exemplary embodiments relate to a connector for connection of a conduit to a vessel of the human body, and, more particularly to a cannula device adapted for attachment to a chamber of the heart and for blood passage therethrough, such as for transporting blood to a ventricular assist device (VAD).

### BACKGROUND

Mechanical circulatory devices (MCDs) such as artificial hearts, ventricular assist devices (VADs) and other blood circulating systems and components have become increasingly recognized as life saving devices for patients whose heart is diseased or has been injured by trauma or heart attack or other causes. VADs in particular, are recognized as a major life saving modality for assisting patients who suffer from congestive heart failure.

VADs must be connected to the natural heart of patients. In order to connect a VAD to the heart of a patient, a conduit assembly is used. The conduit assembly conventionally has a tubular tip body that is inserted into the heart. For proper functioning, the tip body typically penetrates the heart wall to make a connection with the heart through the heart wall. However, various difficulties may present themselves in connecting a conduit assembly with the heart. For example, it is desirable to ensure that there are no leaks through the heart wall in the opening through which the conduit assembly is placed. On the other hand, it is desirable to ensure that tissue from the heart wall does not grow in such a manner to occlude the opening into the conduit assembly. Additionally, it is desirable to obtain uninterrupted flow through the conduit assembly while preventing fluid from stagnating and developing emboli.

For these, and a variety of other reasons, there is a continued desire to provide enhanced methods, systems and devices that will improve the functionality and efficiency of VADs and other similar devices.

### BRIEF SUMMARY OF THE INVENTION

In accordance with the present invention, various embodiments of a cannula assembly are set forth. In accordance with one embodiment, a cannula assembly is provided that comprises a tubular structure coupled with a flange assembly. The flange assembly includes a plurality of wireform loops disposed in a circumferential, woven pattern about an end of the tubular structure. The flange assembly is configured to exhibit a first, collapsed state wherein the plurality of wireform loops extend substantially axially from the tubular structure, and a second, expanded state wherein the plurality of wireform loops extend in a direction having a substantial radial component relative to the tubular structure.

In accordance with another embodiment, another cannula assembly is provided that comprises a tubular structure coupled with a conformal flange. The conformal flange assembly is configured to exhibit a first, collapsed state and a second, expanded state wherein the conformal flange extends substantially radially outward relative to the tubular structure. A proximal surface of the conformal flange is formed of a material that promotes tissue in-growth.

In accordance with yet another embodiment, a method of coupling a cannula to a tissue structure is provided. The method includes collapsing a flange assembly within a delivery member, passing the delivery member through an opening in the tissue structure, and expanding the flange assembly to exhibit a size greater than the opening in the tissue structure. The flange assembly is made to conform to the anatomy of the tissue structure and tissue in-growth between the tissue structure and the flange assembly is promoted.

In accordance with another embodiment of the present invention, a cannula is provided for the transport of blood. The cannula comprises an elongate, flexible tubular conduit defining a blood channel therethrough between a first end and a second end. The conduit is flexible but exhibits a sufficient stiffness to avoid kinking or collapse due to suction forces that may be applied thereto. The first end comprises an adaptor for connection to a heart and a second end is configured for connection to a device such as a blood pump. In one embodiment, the adaptor is resiliently-flexible and assumes an expanded configuration having a wide inlet mouth while in an expanded or deployed configuration. The adaptor is configured to be inserted through incision or other opening within the heart by introduction while in a collapsed or contracted state. Once introduced into, for example, a ventricle of the heart, the adaptor (which may be self expanding) is deployed to assume a desired configuration as a juncture to the heart. In one embodiment, the inflow adaptor assumes a saucer-shaped mouth geometry and is a composite structure comprising a framework of elastic wireforms and a material displaced along the enclosed area wire framework to provide a substantially leak proof conduit.

In an embodiment, there is provided a method to deploy the cannula inlet adaptor in a collapsed or compressed condition and then to expand the prosthesis when it has been moved from the remote location to the location to be installed. In one embodiment, the prosthesis, when deployed, radially exceeds the diameter of the fenestra created within the ventricular wall. According to the present invention, the prosthesis is self-expanding once introduced and deployed. Such a prosthesis is compressed within a constraint provided by an introducer. Once in position, the constraint is removed when the introducer is actuated to disengage the prosthesis and allows the inlet adaptor to resiliently self-expand into a “wide mouth configuration”. In the expanded configuration, the inflow adaptor is preferably in contact with the interior surface (endocardium) of the heart and allows some flex as the heart beats.

According to various exemplary embodiments, the a ventricular cannula may be configured to provide some or any of the following: a distal portion of cannula having an intraventricular conformal flange; a distal portion of a cannula that accommodates varying myocardial wall thicknesses; an intraventricular flange having a normative saucer shaped geometry which is flexible and conformal to variable converging geometry; the ability to attain a conformal interface of an intraventricular flange and myocardium that is substantially insensitive to positional variability; a connection with no significant gaps or crevices around intraventricular flange when secured against variable intraventricular geometry; an intraventricular flange that is minimally traumatic so as to not excessively induce tissue irritation (e.g., no lesion or necrosis); an intraventricular flange with a closed structure preventing tissue and/or pannus growth therethrough; an intraventricular flange that is collapsible without significant permanent deformation in a lumen corresponding to the approximate outside diameter of the conduit portion of cannula; an intraventricular flange that collapses at a force threshold that is greater than that which is anticipated to be experienced when implanted; a cannula device having a distal portion that remains mechanically secured in a conforming orientation with respect to the heart under worst case pushing and pulling applied to the conduit portion of cannula; a device having a distal surface of an intraventricular flange that is substantially smooth and thrombo-resistant; a cannula device wherein a proximal surface of an intraventricular flange comprises texturing, flocking or other features or materials to maintain adhesion to endocardium such as by tissue in-growth; a cannula device having flocking wherein the flocking includes a tight weave or other structure to encourage modest but not excessive adhesion so that the device can be broken free from the tissue at explants (i.e., separates substantially a-traumatically); a cannula device wherein the distal portion of the cannula maintains an adequate fluid seal with the heart under the full range of flow, pressure and loading conditions.

Other features and advantages may be possible, and it is not necessary to achieve all or any of these features or find any of the stated advantages in any embodiment. Therefore, nothing in the forgoing description can or should be taken as limiting.

### BRIEF DESCRIPTION OF THE SEVERAL VIEWS OF THE DRAWINGS

The foregoing and other advantages of the invention will become apparent upon reading the following detailed description and upon reference to the drawings in which:

FIGS. 1A-1D show a front view, a front perspective, a side view and a back perspective, respectively, of a cannula structure in accordance with an embodiment of the present invention;

FIGS. 2A-2D show front views of cannula structures according to further embodiments of the present invention;

FIGS. 3A-3C show a front view, a side view, and a perspective view, respectively, of a connector assembly which may include a structure such as shown in FIGS. 1A-1D or 2A-2D;

FIG. 3D is a sectional view of the assembly shown in FIGS. 3A-3C taken along the section line indicated in FIG. 3A;

FIG. 4A-4E are front, back, side, sectional, and perspective views, respectively, of an embodiment of another cannula structure, with the view of FIG. 4D being taken along section line indicated in FIG. 4A;

FIGS. 5A-5C are front, side and perspective views, respectively, of another embodiment of a cannula structure which includes an integrated conduit portion;

FIGS. 6A-6B are front and side views, respectively, of a single wireform that may be used in association with the structure shown in FIGS. 5A-5C;

FIGS. 7A-7C are side, front and perspective views, respectively, of an adjustable exterior flange according to an embodiment of the present invention;

FIGS. 8A-8C are side exterior, side sectional, and perspective views, respectively, of an assembly that includes the cannula structure shown in FIGS. 5A-5C and the adjustable flange shown in FIGS. 7A-7C;

FIGS. 9A-9B are proximal and distal views respectively of an intraventricular flange of a cannula structure in an expanded configuration and assuming a substantially normative, non-stressed state;

FIGS. 10A-10B are side and front views of the flange shown in FIGS. 9A-9B while in a collapsed configuration with the flange being constrained within a tube segment for delivery of the device through a vessel wall;

FIGS. 11A-11C are perspective, front and side elevation views, respectively, of another assembly that includes a cannula structure having an elbow formed therein along with various components to effect of the cannula coupling between a hollow vessel and a blood flow device;

FIG. 12 is a sectional view of the assembly as taken along section line 12-12 in FIG. 11C;

FIG. 13 is a perspective view of the cannula assembly shown in FIGS. 11A-11C prior to the attachment of hardware for enabling anastomosis to a hollow vessel and for coupling to a blood flow device;

FIG. 14 is a perspective view of a support collar that may be used to assist the coupling of a cannula structure to a hollow vessel according an embodiment of the present invention;

FIG. 15 is a perspective view of an adjustable restraint that may be used to assist the coupling of a cannula structure to a hollow vessel according an embodiment of the present invention; and

FIG. 16 is a side view of another assembly in accordance with another embodiment of the present invention.

### DETAILED DESCRIPTION OF THE INVENTION

As utilized herein, terms such as “about”, “approximately”, “substantially” and “near” are intended to allow for tolerances that are acceptable in the industry.

Various embodiments are described more fully below in sufficient detail to enable those skilled in the art to practice the claimed invention. However, embodiments may be implemented in many different forms and should not be construed as being limited to the embodiments set forth herein. Aspects of one described embodiment may be combined with aspects of other embodiments. The following detailed description is, therefore, not to be taken in a limiting sense.

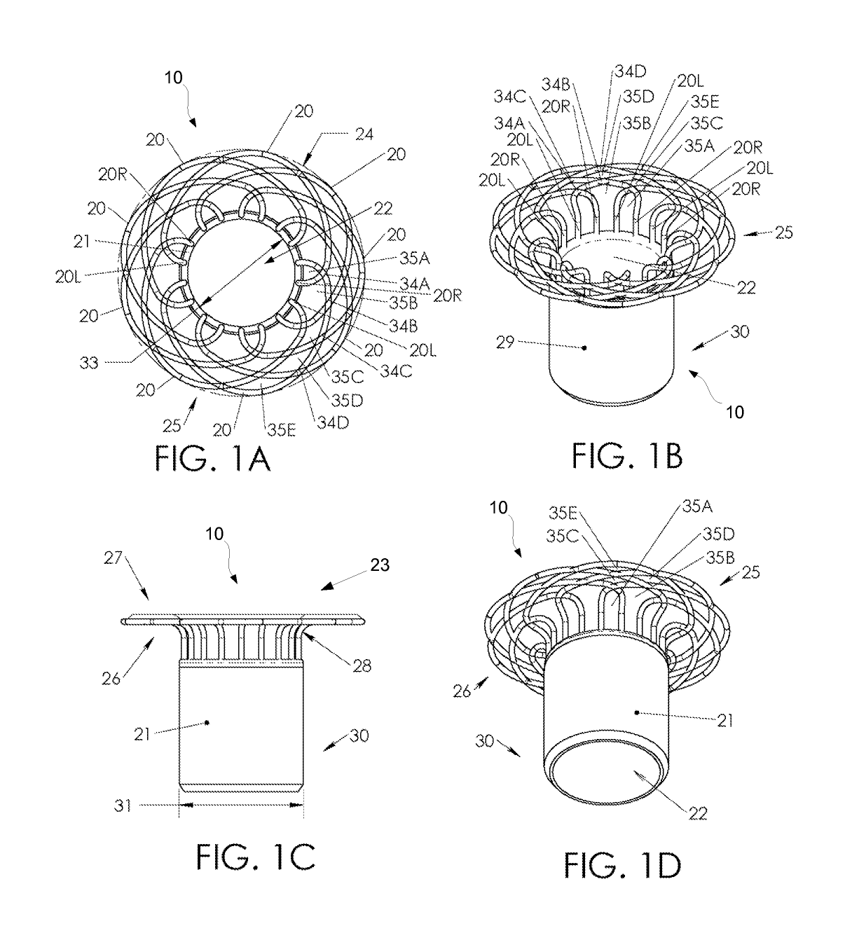

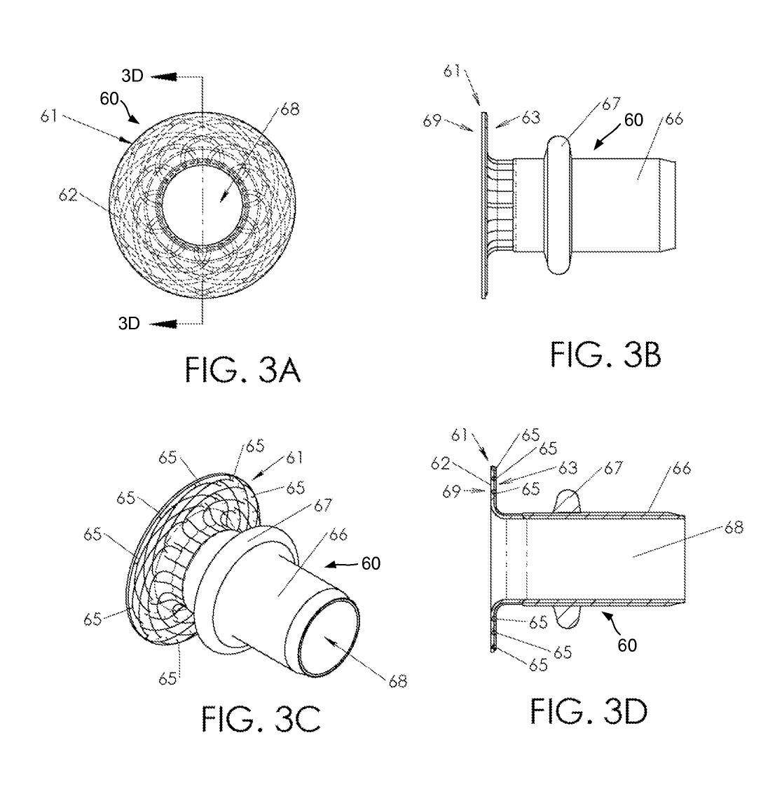

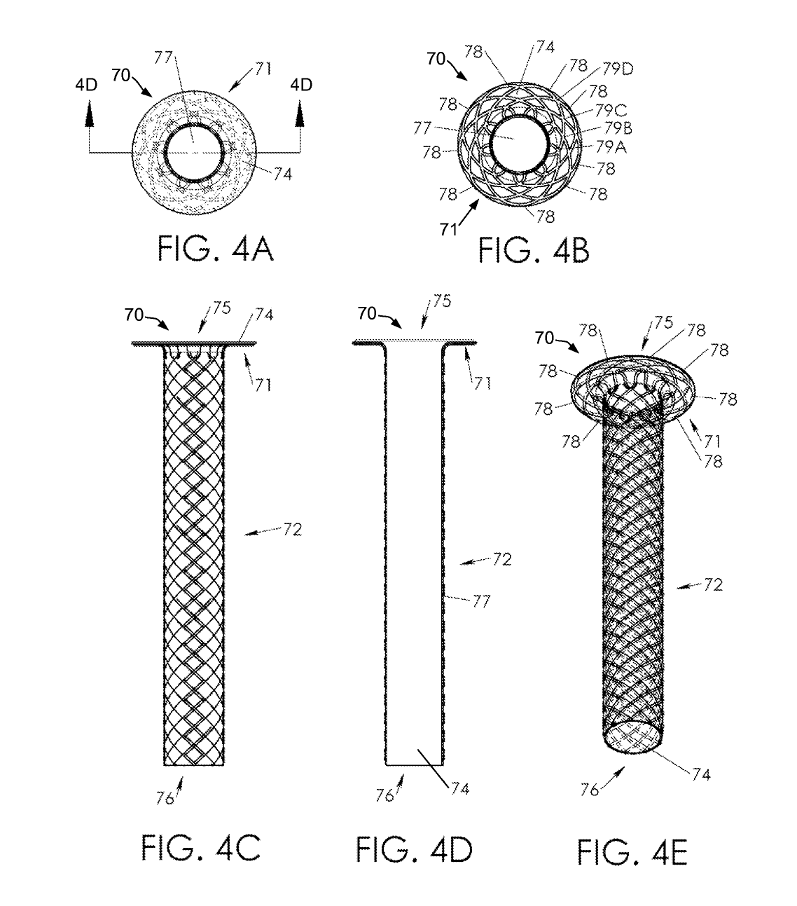

Referring to FIGS. 1A-1D a first embodiment of the cannula frame assembly 10 is shown. The cannula frame assembly 10 includes of an array of interwoven wires or wireforms 20 that are shaped and positioned relative to one another so as to form a flange assembly 25 at the distal end 23 of the assembly 10. The assembly 10 also includes and a tubular support base 21 at the proximal end 30 of the assembly that is sized and configured to support the wireforms 20. The tubular support base may be formed of a relatively rigid material that assists in maintaining the flange assembly 25 in a desired shape or geometry whether the flange assembly 25 is in a deployed state or in a collapsed state for delivery through a vessel wall. The cannula frame assembly 10 may be a foundational component of a larger assembly, with materials and other features being added to both the interior and exterior depending on the device application. For example, additional features will be described in greater detail in reference to FIGS. 3A-3D and FIGS. 4A-4E.

In one embodiment, the tubular support base 21 may be configured to define a lumen 22 exhibiting an internal diameter 33. The tubular support base 21 also exhibits an external diameter. In one embodiment, the flange assembly 25 is configured to also partially define lumen 22 and exhibit similar diametrical dimensions. In the illustrated embodiment, the flange assembly 25 comprises a circular pattern of wireforms 20 with each wireform 20 being configured as a loop extending from a left axial segment 20L to a right axial segment 20R. The left axial segment 20L and the right axial segment 20R are each connected to the tubular support base 21 at spaced apart radial positions. As shown in FIGS. 1A-1D the assembly of multiple wireforms 20 that loop along a radially outward path provide an expanded flange assembly further characterized with radial bend 28, a distal face 27 and a proximal face 26.

Tubular support base 21 may by formed using a number of different processes from a variety of different materials. For example, it could be a machined metal or plastic piece having axial holes formed therein for receiving the axial segments 20R and 20L of wireforms 20. In another embodiment tubular support base 21 may include an over molded polymer material such as polyester, PTFE, Polyurethane, PEEK or other biocompatible thermoplastic. Additionally, the wireforms 20 may be secured to the support base 21 by a variety of techniques such as, for example, brazing, welding, interference fit or by an adhesive material. Of course, the manner of coupling the wireforms 20 to the support base may depend, at least in part, on the materials being used to form each of the components.

In the embodiment illustrated in FIGS. 1A-1D, the cannula frame assembly 10 includes nine individual and discrete wireforms arranged in a symmetrical circular pattern. In such a configuration, the right axial segment 20R of a given wireform 20 is positioned approximately 180 degrees apart from the left axial segment 20L of each wireform 20. Each wireform 20 is woven with respect adjacent wireforms 20 resulting in crossings 34A-34D and gaps 35A-35D among the plurality of wireforms. The radial innermost crossing or set of crossings exhibited by the wireforms 20 (i.e., those closest to lumen 22) may be referred to as the primary crossings 34A. In the presently illustrated embodiment, there is also a second set of crossings 34B, a third set of crossings 35C, and forth set of crossing 34D, progressing radially outward with the fourth set of crossings 34D being the radially outermost crossing. With respect to the gaps 35A-35D between wireforms 20, gap 35A and gap 35B are positioned between the inner diameter 33 and the outer diameter 31 of the support base 21. Additional gaps 35C, 35D, and 35E, located at progressively radial outward positions, are approximately diamond shaped and are dependent on the number, shape, and positioning of wireforms 20 used.

Due to the size and shape that each wireform 20 assumes, and because the wireforms 20 are woven, the flange assembly 25 acts as an integrated structure where no wireform 20 acts independently of the other wireforms 20. A benefit of the frame assembly as embodied is the uniformity of the resulting structure with the crossings 34A-34D being distributed in a spread apart relationship and the gaps 35A-35E not varying substantially in size, especially with respect the outward diamond shaped gaps 35C-35E.

The frame assembly 25 provides a flexible framework for the cannula device so that it may adapt and conform to varied anatomical geometry when attached to a hollow vessel of a patient, such as the ventricle of a heart. The flange assembly 25 provides an appropriate combination of flexibility and bias so as to assume a variable deflected geometry and conform to abutting surfaces within the hollow vessel (e.g., the interior surface of a ventricle). This helps to eliminate, or at least minimize, potential gaps that might otherwise develop between the proximal face 26 of the flange assembly 25 and the tissue of the hollow vessel in which the cannula assembly 10 is implanted.

Additionally, the flexibility and configuration of the distal flange assembly 25 enables it to be radially collapsed within a lumen of a delivery device (e.g., a catheter). For example, such a lumen may exhibit a cross-sectional geometry that approximates the outside diameter 31 of tubular support base 21. When the flange assembly 25 is compressed radially, it elongates axially and the wireforms 20 assume a deflected geometry, whereas the gaps 35A-35E change shape as the angles associated with crossings 24A-24D changes.

To enable the transition between a radially collapsed configuration (e.g., within a delivery device) and a radially expanded configuration (e.g., implanted within a hollow vessel), the wireforms 20 may include a material enabling sufficient deformation so the permanent strain induced when flexing wireforms 20 is minimal. One material may include a super-elastic metal alloy such as Nitinol (a nickel-titanium alloy). A further advantage of Nitinol is that the wireform 20 can be formed precisely by shape training nitinol wire at elevated temperatures as will be appreciated by those of ordinary skill in the art. However, other metals or polymers may be used if capable of recovering from large deformations. It is not necessary that the wireforms 20 exhibit a circular cross section or be wire formed into the desired geometry. For example, such could be formed from a polyester strip of a rectangular or other polygonal cross section which is molded into the intended loop geometry.

The thickness of flange assembly 25 in the illustrated embodiment is approximately twice that of the thickness of wireforms 20. In one embodiment, a frame assembly exhibiting an approximate lumen diameter of 5-6 mm may include circular Nitinol wire exhibiting a diameter the range of approximately 0.25 mm to approximately 0.40 mm resulting in flange assembly thickness of approximately 0.5 mm to approximately 0.8 mm as measured at crossings 34A-34D. Thus, in such an embodiment, the flange need not exceed a thickness of 1 mm. The ability to support the flange assembly 25 with wireforms 20 of relatively small diameter helps to enable the flange assembly 25 to collapse within a small cross sectional area while also providing sufficient strength and flexibility to act as a conformable flange when implanted. In one particular example, it has been determined that the when the outside diameter 24 of the flange assembly is approximately 15 mm, it may be efficiently collapsed and loaded within a lumen exhibiting a diameter of approximately 7 mm—which is of less than half the expanded diameter.

As indicated above, the cannula frame assembly 10 may be delivered in a collapsed configuration through the lumen of a delivery device. The conformal flange assembly 25 may be introduced through a small lumen and deployed to assume the larger profile shown in FIGS. 1A-1D. In the case of implanting the cannula frame assembly 10 to a ventricle of the heart, the shoulder of conformal flange assembly 25 rests against endocardium such that there is little or no crevice or gap between endocardium and the proximal side 26 of the flange. Such enables a substantially wide mouth receptacle to be provided exhibiting an outside rim diameter of twice (or greater) than that of the inner lumen 22. The interface of the cannula with a hollow vessel may be considered as being analogous to a sink drain where fluid flows toward and enters the conduit at the bottom of the basin (i.e., ventricular apex).

In certain embodiments, the flange assembly 25 may be configured to exhibit a substantially conical shape, a flared shape, or some other shape such that the angle associated with bend 28 is greater than 90 degrees. Further, the flange assembly 25 may exhibit a curved geometry such that distal surface 27 and proximal surface 26 are not substantially flat but are convex or concave. In the case of interfacing with relatively large vessels or cavities, a saucer-shaped profile may be utilized that is essentially completely radial enabling it to adapt to the surfaces of cavities that are substantially large and open. Moreover, the wireforms 20 of the flange assembly 25 may be biased backward at an angle corresponding to bend 28 and may be less than 90 degrees. This ensures that flange assembly 25 is always in a stressed and deflected state for the purpose of eliminating any crevices that might otherwise be exhibited around proximal face 26. While not limiting, it is contemplated that the angle associated with bend 28 may be within the range of approximately 60° to approximately 115° (as measured between the radial outer surface of the support base 21 and the proximal surface 26 of the flange assembly) for most applications.

The cannula frame assembly 10, when implanted within a patient's ventricle, may also have the effect of stenting the interior walls of the ventricle away from the inlet at proximal end. Thus, it can prevent or reduce the chance of the septum of the heart from encroaching across the inlet and occluding inflow through the lumen 22. The cannula frame assembly 10 also prevents, or at least reduces, the risk of ventricular collapse when a suction force is applied to the ventricle by the cannula.

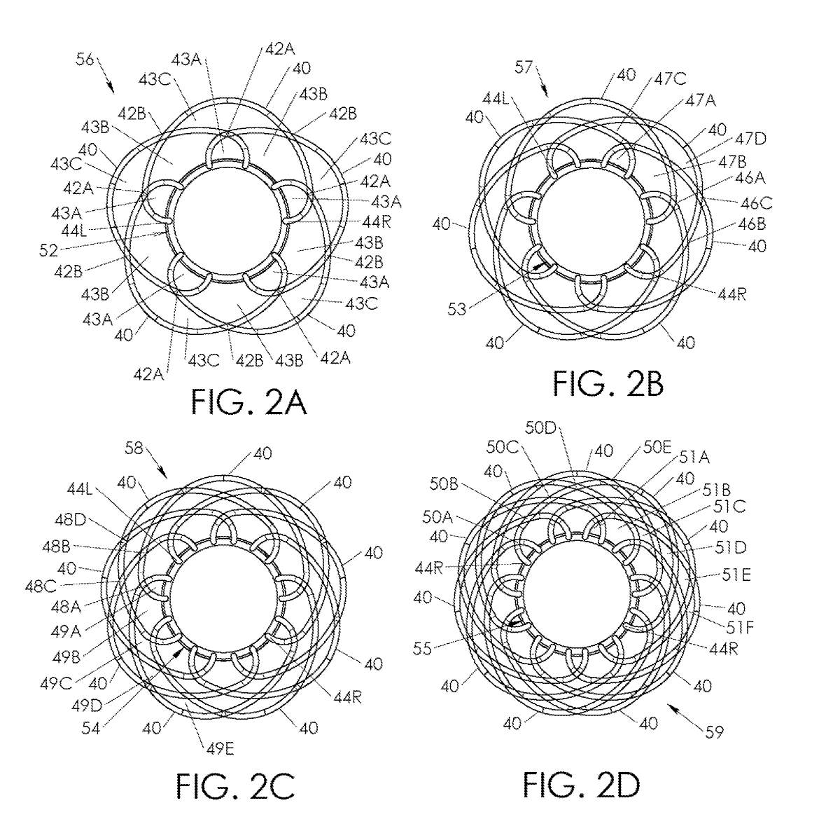

Referring now to FIGS. 2A-2D, a front view is shown of various flange assemblies 56, 57, 58 and 59) that similar to the flange assembly 25 described with respect to FIGS. 1A-1D, even including a similar overall flange diameter and lumen, but utilizing a different number of wireforms 40.

FIG. 2A shows a flange assembly 56 comprising five wireforms 40 with tubular base structure 52 coupled to ten axial segments 44L and 44R of wireforms 40. Corresponding to this configuration there is a first set of crossings 42A, a second set of crossings 42B, a first set of gaps 43A, a second set of gaps 43B, and a third set of gaps 43C (each set of crossings or gaps having a quantity of five). On account of the fewer number of wireforms 40 as compared to FIBS. 2B-2D, there are fewer crossings 42A and 42B and gaps 43A-43C. Additionally, the gaps 43A-43C are larger as compared to other configurations utilizing a greater number of wireforms. Correspondingly, if the same wire diameter is used for the various embodiments of FIG. 2A-2D, the flange assembly 56 will be less stiff than the other embodiments.

The flange assembly 57 shown in FIG. 2B includes seven wireforms 40 with the tubular support base 53 being coupled to fourteen axial segments 44L and 44R of wireforms 40. This configuration results in three sets of crossings 46A, 47A and 48A, each set including seven crossings. It is noted that, while the five-wireform configuration shown in FIG. 2A has two circular patterns of five crossings for a total of 10 crossings, the seven-wireform configuration shown in FIG. 2B comprises three circular patterns of seven crossings for a total of twenty-one crossings. Thus, by adding two additional wireforms 40, the number of crossings more than doubles from the five-wireform embodiment to the seven-wireform embodiment. This also results in many more, yet smaller, gaps 47A-47D.

As shown in FIG. 2C, the flange assembly 58 comprises nine wireforms 40 with the tubular support base 54 being coupled to eighteen wireform segments 44L and 44R. Again, an increased number of crossings 48A-48D can be seen with an increased number of smaller gaps 49A-49E.

The flange assembly 59 shown in FIG. 2D includes eleven wireforms 40 with the tubular support base 55 being coupled to twenty-two axial segments 44R and 44L of wireforms 40. Again, this results in even more crossings 50A-50E and more, yet smaller, gaps 51A-51F. By altering the number of wireforms 40 the mechanical characteristics of the flange assembly can be altered within the same basic geometric constraints. A practical limit is encountered when the number and density of the wireforms 40 is increased to the level that the flange assembly becomes too stiff. This can be countered, to a degree, by using finer wire to form the wireforms. Thus, it is also possible to get approximately the same composite stiffness using a greater number of wireforms exhibiting a smaller cross-sectional area (e.g., diameter) as a composite stiffness of a flange assembly comprising few numbers of wireforms exhibiting a larger cross-sectional area (assuming the same cross-sectional shape being used in the wireforms).

Thus, various configuration are contemplated and the flange assembly may be designed to distribute the pressure exerted against the tissue (by the proximal face of the flange assembly) over an increased surface area so as to reduce or eliminate stress concentrations that could cause an inflammatory response or otherwise harm the tissue.

In the illustrated embodiments of FIGS. 2A-2D, the wireform patterns, which correspond to an incrementally finer mesh, utilize an odd number of wireforms. Also, in each case the relative position of the right axial segment of each wireform is approximately oriented 180-degrees from the left axial segment of each wireform. However, other configurations are contemplated, including other numbers of wireforms and there position of coupling with their associated support base. It is also understood that each wireform need not be a discrete segment of wire but that multiple loops could be formed along the lumen of the cannula so that the flange assembly comprises as few as one continuous wire.

Referring to FIGS. 3A-3D, the ventricular interfacing portion of a cannula assembly is shown including an enclosure structure or member 62 associated with the flange assembly 61 as well as a sewing ring 67 that may be used to suture the cannula assembly to the tissue around an anastomosis site.

The enclosure 62 provides a blood contact surface on distal face 69 of the cannula assembly 60 and may be considered as forming webbing between gaps of wireforms 65. The enclosure 62 may include an elastic material that can withstand sufficient elongation and deformation and may be characterized with the ability to dramatically flex and so as to not cause an excessive increase of the stiffness of flange assembly 61 while it transitions from a collapsed state to a deployed state. Additionally, the enclosure should be formed of a material that will resist tearing or detachment from wireforms 65 during such transition. Some examples of materials that may be used to form the enclosure include segmented polyurethanes, silicones, or expandable ePTFE.

Example of suitable biocompatible polyurethanes include Biomer (World Heart Inc.), BioSpan (DSM Biomedical) and CronoFlex AR (AdvanSource Biomaterials). Biomer is particularly well suited to the application due to extended flex life, high elongation properties, and low stiffness. It has been utilized in long life applications including ventricular assist device (VAD) components such as bladders that are adapted for containing blood and subject to repeated flexure. In another embodiment, an implantable silicone may be utilized to form the enclosure 62. One particular example includes non-restricted silicone dispersions available from NulSil Technologies Inc. Such silicone is typically of higher elongation and of lower stiffness than polyurethane materials but generally does not exhibit as good blood compatibility as the segmented polyurethanes such as Biomer.

In utilizing an elastomer such as dispersion of silicone or polyurethane, the enclosure 62 can be formed by various methods. One method includes dip molding the elastomeric material on a mandrel to form a sleeve having a desired internal geometry and an external geometry that will substantially match or conform to the internal geometry of the cannula frame structure along lumen 68 and flange assembly 61. Once the sleeve is formed and positioned within the cannula frame assembly, additional application(s) of the dispersion of elastomeric material can be added to the frame assembly 60 to adhere the preformed sleeve to the frame assembly. In another embodiment, the elastomer dispersion can be applied directly to the frame assembly without performing a sleeve. This may be accomplished by supporting the frame assembly 60 on a mandrel and dipping it in the elastomeric dispersion or by applying it in some other manner such as pouring, brushing or spraying to essentially over mold wireforms 65 and coat internal lumen 68 of tubular support base 66.

For an application such as a ventricular connector for attachment to the heart, it may be desirable that the elastomeric material be continuous and non-interrupted within the interior of cannula so as to be smooth with no voids, providing a leak proof conduit for the passage of blood. To improve the blood compatibility of the internal surfaces of the device it may be of benefit to subsequently modify the blood contacting surfaces by adding a secondary coating to the substrate or foundation of the enclosure 62.

According to one embodiment, an internal blood contacting portion of enclosure 62 is provided by an expanded polytetrafluoroethylene (ePTFE) sleeve affixed to the inside of frame assembly 60. Grafts formed of ePTFE are widely used in the art for providing blood conduit as they have a micro-porous structure that facilitates the formation of a controlled biological layer on the surface. A tubular ePTFE sleeve may be flared at one end to match the geometry of flange assembly 61 so that the ePTFE provides a surface covering all the gaps and crossings of the wireforms 65 making the flow path of blood substantially seamless. An example of suitable ePTFE sleeve material is sold under the trade mark Aeos by ZEUS. The ePTFE may be attached using various methods. One example includes thermal bonding fluoropolymer sleeves through the gaps formed by wireforms 65, such that the wires of flange assembly 61 are embedded between two sleeves of a fluoropolymer. For example, an ePTFE internal sleeve may be bonded to a fluorinated ethylene propylene (FEP) outside sleeve or spiral wrap. In another embodiment, the ePTFE sleeve may be etched to improve adhesion to a subsequently applied elastomeric adhesive for attachment to flange assembly 60. Another method of joining an ePTFE sleeve includes sewing and/or tying it to the cannula frame at strategic positions using a suitable suture material such as filaments of ePTFE. Yet another method may include mechanically fastening an ePTFE sleeve to the cannula frame using eyelets, clips, rivets or the like.

Still referring to FIGS. 3A-3D, the portion of tubular support base that interfaces with myocardial wall or other tissue may also include an outer textured surface (not specifically shown). Likewise, the outer surface of at least a portion of flange assembly 61, such as along the proximal surface 63 of flange assembly 61, may include a textured surface. Such textured surface may encourage the in-growth of tissue when the device is implanted. Additionally, the surfaces exposed to tissue may be formed of materials having desired properties (e.g., porosity) that will enhance tissue growth and attachment to the device. Such in-growth of tissue may provide better bonding between the patient and the cannula and, further, may reduce risk of infection.

A sewing ring 67 may be provided at the appropriate position along tubular support base 66 for suturing the cannula to the tissue surrounding the exterior of the anastomosis site. In one example, the sewing ring 67 may be constructed of velour or plastic. If the sewing ring 66 is constructed of a relatively hard or rigid material (such as relatively rigid plastic), the sewing ring 66 may include suture holes. However suture holes may not be necessary if the sewing ring is constructed of a substantially pierce-able material, such as polyester velour. The sewing ring may be able to form an apical shape to conform to the corresponding surface of the heart which the sewing ring engages. Such sewing rings are known in to those of ordinary skill in the art and can be utilized in various configurations. Although the sewing ring 67 is shown to have a low circular profile, it may exhibit a larger flange geometry including a flange that is of the same size as, or larger than, the flange assembly 61.

In one embodiment, a locking nut and sewing ring may be mounted near the proximal end of the tubular support base 66 to effect fixation to the outside surface of the heart. The cannula assembly 60 may be attached to the heart by first coring a suitable sized hole (or cutting a suitable size incision) in the apex of the left ventricle (or another suitable location) and then inserting the cannula assembly 60, with the flange assembly 61 in a collapsed state, into the aperture of the heart. A sewing ring 67 may then be slid up the cannula assembly 60 until it contacts the heart. The sewing ring 67 is snared around the stem of the cannula assembly 60 and then sewn to a ring of pledgets placed around the base of the ventricular apex. The adherence of the pledgets to the myocardium may be augmented, for example, by the use of fast curing glue.

In another embodiment, the cannula assembly 60 may include a structure that engages a cooperating interface (not shown) mounted on a sewing collar, where the sewing collar is connected to a sewing ring. Accordingly, the axial position of the sewing collar can be adjusted on cannula assembly 60 providing for the adjustability of the sewing ring location. It is also further envisioned in another embodiment that silicone adhesive may be used to fix the sewing collar in the desired position.

The distal portion of the cannula assembly in the embodiments described above is configured to interface with the ventricle and provide a biological exit port for blood flow. In most applications it may also be desired to provide a flexible conduit portion for transporting blood from the ventricle of the heart to a remote location at a position away from the anastomosis site. The path the conduit portion must take for optimal anatomical fit of the VAD may be a relatively tortuous path requiring the conduit portion of the cannula to assume tight bends while also being substantially kink and collapse resistant.

In one embodiment of the invention, the cannula may include a reinforced graft with a spiral structure. The spiral structure may include a helical metal wire, a beaded (or grooved) plastic as is widely practiced in the art. As it is desirable to add connections along the flow path, wherein, for example, an intermediate adaptor would be required to attach the conduit portion of the cannula assembly with the ventricular interface portion of the cannula assembly, the blood contacting sleeve may be used for covering distal face 69 of flange assembly 60 and lumen 68 of tubular support base 66 to extend beyond tubular support base and to embody a conduit of sufficient length to transport blood to, for example, a pump at a remote location. The conduit portion of the sleeve could be reinforced with an embedded wire coil or supported externally with beading that is mechanically coupled to tubular support 66 to provide a seamless integrated cannula when a lengthy flexible conduit portion is required to extend from proximal end of the ventricular attachment portion of the cannula assembly.

Referring now to FIGS. 4A-4E, an embodiment of an extended length cannula is provided whereas wireforms 79 woven together at the distal end 71 in flange assembly 75 are also woven down the conduit portion 72 of cannula 70 to proximal end 76. In this configuration, a separate tubular support base is not necessary since the weave of wireforms 78 provide sufficient radial reinforcement to support the flange assembly 71 to interface with variable geometry within the hollow vessel. As seen in FIG. 4B, the flange assembly 71 may include similar weave geometry as previous described with crossings 79A-79D extending radially outwardly from the lumen 77. However, unlike prior embodiments, many crossings are also exhibited along conduit portion 72 of the cannula assembly 70, providing reinforcement along the conduit portion 72. An enclosure structure 74 is further provided along the distal face 75 of flange assembly 71 and through lumen 77 to provide a blood conduit and enclose or cover the gaps defined by the weaving of the wireforms 78.

The function and methods to attach the enclosure structure 74 are consistent with the enclosure 62 of FIGS. 3A-3D. The embodiment illustrated in FIGS. 4A-4E include wireforms 78 sufficiently supported within flange assembly 71, while the conduit portion 72 exhibits a minimal radial thickness, an extended length, and flexible yet reinforced. Additionally, the internal surfaces of lumen 77 are substantially seamless in incorporating the enclosure structure 74 through the entire length of the cannula assembly 70. Thus, it should be appreciated that in many applications an intermediate tubular base structure for supporting wireforms may be a concern as this increases the radial thickness of the conduit portion and makes it more challenging to provide a seamless, flexible cannula of extended length.

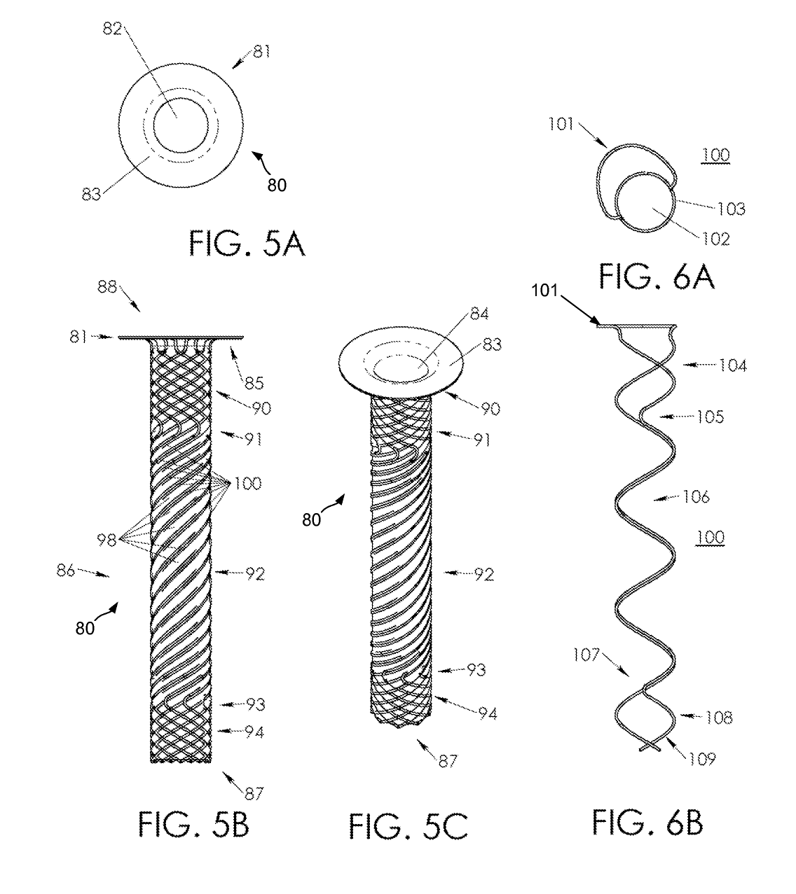

FIGS. 5A-5C illustrate another embodiment of a cannula frame assembly 80 incorporating a wire framework that extends from distal end 88 to proximal end 87, defining an elongated conduit portion 86. Differing from the embodiment shown in FIGS. 4A-4E, cannula frame assembly 80 includes a helical coil portion 92 displaced between a distal woven portion 90 and a proximal woven portion 94. The helical coil portion 92 provides a section of the cannula frame assembly 80 having helical gaps 98 that enable the device to be more easily bend and form a tight bend radius without kinking than does a conduit assembly featuring no helical portion. A transitional portion 91 extends from the distal woven portion 90 to the helical portion 92. Another transitional portion 93 extends from the proximal woven portion 94 to the helical coil portion 92.

The cannula frame assembly 80 includes a circular pattern of wireforms 100 that are woven together forming the tubular framework of the device in forming lumen 82. A flange assembly 81 may be included at distal end 88 similar to other embodiments previously described. The cannula frame assembly 80 may further comprise an enclosure material along distal surface 83 and interior surface 84 of appropriate biocompatible proprieties for the passage of blood.

Referring now to FIGS. 6A-6B, front and side views, respectively, of a single wireform 100 are shown. The wireform 100 may be used for constructing the cannula frame assembly 80 of FIGS. 5A-5C. The wireform 100 may be formed from a shape memory alloy, such as Nitinol. For example, a Nitinol wire may be shape trained in the shown geometry. The wireform 100 includes a flange loop 101, a counter winding portion 105, a transitional portion 105, a helical coil portion 106, a transitional portion 107, another counter winding portion 109, and terminal ends 109 that correspond to proximal end 87 of cannula frame assembly 80. As shown in front view of FIG. 6A, the wireform 100 includes a flange loop 101 at the distal end but is predominately a tubular structure 103 encompassing a central opening 102 along the conduit portion of the wireform.

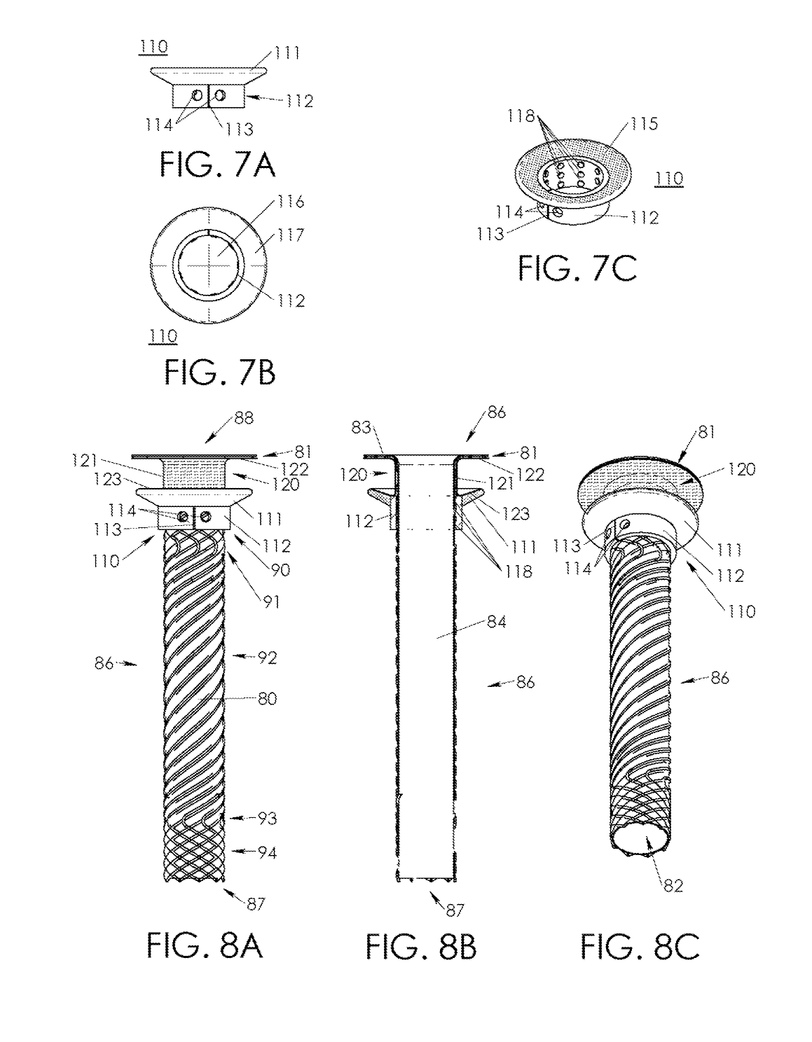

FIGS. 7A-7C show side, front, and perspective views, respectively, of an external fixation flange according to a preferred embodiment of the present invention. The external fixation flange assembly 110 includes a restraining flange 111 connected to base collar 112. Restraining flange 111 is adapted for abutting against exterior tissue at the anastomosis site and includes a distal surface 115 configured for tissue contact and homeostasis. Distal surface may be of a porous material to facilitative tissue adhesion and preferably is flexible to accommodate slight circumferential variations in thickness for facilitating hemostasis and so that pressure against tissue around the anastomosis site can be well distributed to avoid necrosis.

The base collar 112 is configured to be adjustable in its position along the conduit portion of the cannula frame assembly. The base collar 112 defines a lumen 116 in which retention projections 118 are disposed along its interior surface. The base collar 112 further comprises a slit 114 and separator holes 114 for enabling the lumen 116 to be increased in radial size for adjusting position of the external fixation flange assembly 110.

FIGS. 8A-8C show the cannula frame assembly 80 of FIGS. 5A-5C with an external fixation flange assembly 110 attached thereto and also with a textured overlay 121. The textured overlay 121 includes a tubular segment 120, a distal flanged portion 122 and proximal flanged portion 123 and is thus disposed around all surfaces expected to be in contact with tissue around the anastomosis device. The textured overlay 121 may include a compliant material that facilitates tissue in-growth so that the cannula assembly will exhibit sufficient adhesion around the anastomosis site. The textured overlay 120 may also be configured to aid in rapid hemostasis at the time of implant and to prevent significant leakage of blood under various loading conditions over the service life of the device.

According to one embodiment, a distal flange portion 122 of the textured overlay 121 is affixed to the proximal face 85 of flange assembly 81 and at least a portion of the tubular s 86 of the frame assembly 80 corresponding at least to the length of the device extending through a tissue wall at an anastomosis site. The textured overlay 120 may sized and positioned so that the fixation flange assembly 110 may remain axially adjustable to providing a secure coupling despite variations in wall thickness at the anastomosis site (depending, for example, on the position of the cannula and that anatomy of the patient).

Considering FIGS. 8A-8C in light of FIGS. 7A-7C, it will be appreciated that external fixation flange assembly is adapted for fitting along distal woven portion 90 of tubular section 86 of the assembly 80. Projections 118 may be configured to fit within gaps defined by the wireform frame work along the woven tubular section 90. On account of there being several alignment positions along the woven tubular section 90, the relative position of the flange assembly 81 and restraining flange 111 may be adapted or adjusted to accommodate a number of tissue thicknesses.

The collar base 112 may include a substantially resilient material to further effect a close fitting relationship with and effective coupling between the fixation flange assembly 110 and the radial exterior surface of the distal woven section 90, thereby preventing the external fixation flange assembly 110 from slipping along the distal woven portion 90 of the frame assembly 80. For the purposes of adjustment, separator holes 114 may be provided so that a spreader tool (not shown) may engage the fixation flange assembly 110 and increase the width of slot 114 such that the opening 116 is temporary enlarged. This enables the axial position of external fixation flange assembly 110 to be adjusted so that tissue around the anastomosis site is subject to a sufficient, but not excessive, compression force for the purposes of locking the device in position and obtaining a substantially crevice-free interface along the proximal face 85 of flange assembly 81.

Although this is just one way of providing an external flange that is axially adjustable, other structures and assemblies are envisioned including clamping to a reinforced portion of the conduit portion of the cannula or with the incorporation if interfacing members that may include a threaded, ratchet, or bayonet type interconnection between the frame assembly and the external fixation flange assembly.

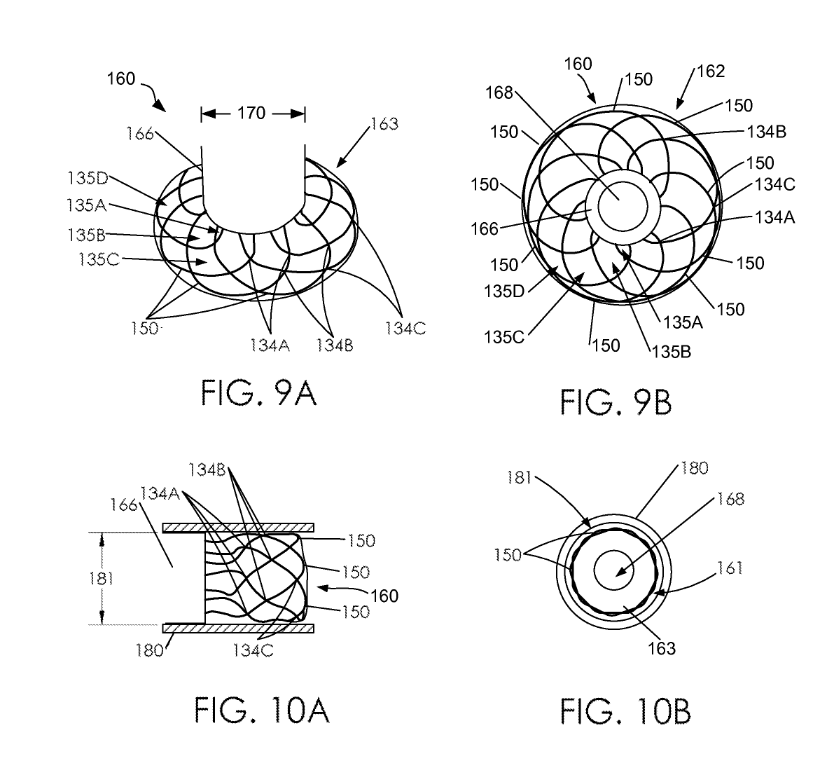

Referring now to FIGS. 9A, 9B, 10A and 10B, the flange assembly portion of a cannula device is shown in various expanded and constrained states. With respect to an expanded configuration, FIG. 9A is a perspective view of the proximal side of the cannula assembly 160 and FIG. 9B is a distal view of cannula frame assembly 160. The cannula assembly 160 includes a tubular base portion 166 supporting an array of woven wireforms 150 that from a flange assembly 161 in a similar manner as described in reference to FIGS. 1A-1D. In the normative expanded configuration, crossings 134A-134C and spacing 135A-135D are configured in a radial outward projection normal to the symmetrical axis of the device. Gaps 135A-135D are covered with enclosure 163 of an elastomeric material so that flange assembly 161 functions as a closed composite structure that can flex and conform to variations in intraventricular geometry around the anastomosis site yet maintain sufficient stiffness to be retained with the hollow vessel under normative loading conditions without collapsing. It will also be appreciated that the composite nature of the device permits substantial deformation of the wireform portion so that it can be delivered through an opening of minimum size.

As seen in FIGS. 10A-10B, the flange assembly 161 is shown in a collapsed state with the cannula assembly loaded within a tubular delivery member 180 having an internal diameter 181 that is approximately the same as the outside diameter 170 of tubular base portion 166. In enabling collapse of the flange assembly 161 without permanent damage to wireforms 150 and elastomeric enclosure 163, the distal portion of the device transitions from a radial-outward flange configuration to a tubular configuration when collapsed (i.e., in the delivery state shown in FIGS. 10A and 10B). Accordingly, wireforms 150 bend and crossings 134A-C and spaces 135A-135D become radially displaced to a substantially common radial distance relative to a longitudinal axis of the cannula assembly 160. This results in a substantially tubular structure with the flange assembly 161 being temporally constrained in a stressed state for delivery of the device through a vessel wall. When delivery member 180 is proximally withdrawn relative to the cannula device 160, the flange assembly 161 self-expands to assume the expanded geometry shown in FIGS. 9A-9B without a significant degree of permanently induced strain.

As seen in FIG. 10B, the lumen 168 is substantially preserved when the distal end of the device is collapsed. Thus, an introducer tool may extend through lumen of the entire device to access the vessel wall to assist, for example, in making an incision or otherwise effecting deployment of the cannula assembly 160. Thus, the minimal radial thickness of the cannula assembly 160 as embodied in a composite structure provides numerous benefits and advantages including that of enabling the device design and methodology to be adapted for minimally invasive surgery.

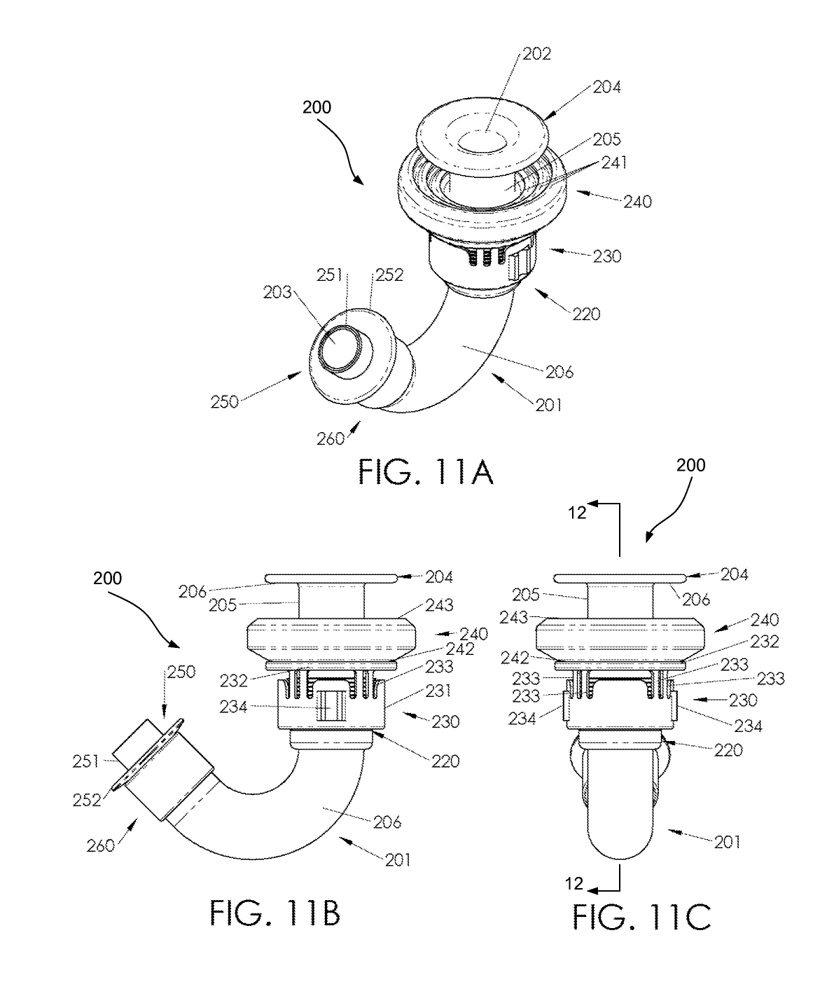

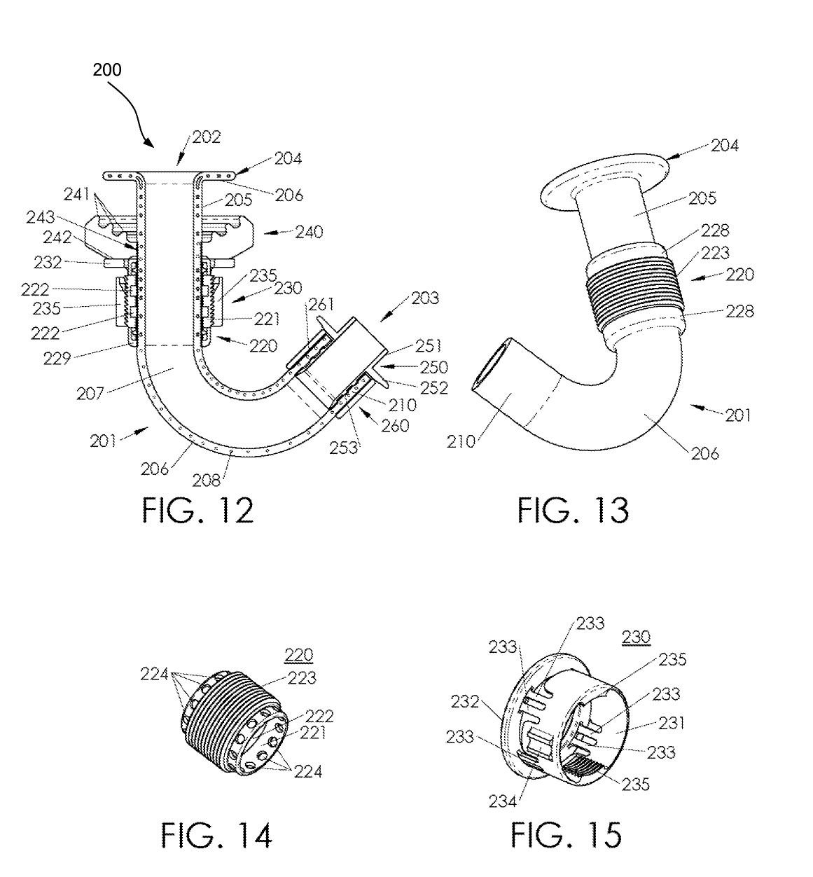

FIGS. 11A-11C and 12-15 show a cannula assembly 200 according to another embodiment of the present invention. The cannula assembly 201 includes an elbow portion 206 and various components that enable the device to be secured to a hollow vessel on the distal end 202 and attached to a blood flow device at the proximal end 251. FIG. 12 shows a sectioned elevation view of the cannula assembly 200. FIG. 13 shows the cannula base assembly apart from additional components used for attachment of the assembly to the heart and to a blood flow device. FIG. 14 shows a support collar and FIG. 15 shows an adjustable restraint according to one particular embodiment.

Referring to FIGS. 11A-11C, the cannula assembly 200 includes a base assembly 201 as a tubular structure or conduit for blood flow from the distal end 202 to the proximal end 203 of the cannula assembly 200. The base assembly 201 comprises an intraventricular flange or flange assembly 204 which may be compliant for deployment in a collapsed configuration through a vessel wall as described in reference to previous embodiments. The base assembly 201 includes an elbow portion 206 between two substantially straight tubular sections 205 and 210. While the previously described embodiments include a structure that enables an acute bend to be formed from a substantially unstressed straight tubular member without kinking, the device shown in FIGS. 11A-13 includes a cannula having a predetermined bend in which the normative unstressed geometry is of a desired curvature. This curvature may be particularly advantageous for use with certain blood flow devices in which a dramatic redirection of flow is required for optimal device placement within a confined space within the patient.

By providing an integrated bend geometry into the construction of the cannula assembly 200, minimal stresses are induced into the cannula assembly 200, the tissue to which it is attached, as well as any blood flow device that is to be attached to proximal end 203. This is especially beneficial when it is needed to redirect the flow in excess of 90°. For example, FIG. 11B shows a bend angle that is in the range of approximately 135°. In one embodiment, the conduit portion of the cannula assembly 200 may be provided with a nominal bend by first forming a straight woven wireframe structure (such as that illustrated in FIGS. 4A-4E) made from a shape memory alloy such as Nitinol. Prior to over molding and embedding the wireframe in an elastomeric material or other enclosure, the wireframe structure may be supported over a bent rod or mandrel of the desired curvature. The diameter of the mandrel for shape setting the bend may be approximately the same as the internal diameter of the wireframe structure. The wireframe structure is then shape set in a furnace so that it will exhibit the bend curvature in a non-stressed state.

After shape setting the woven wireframe, the over molding of the elastomeric material can then be accomplished by supporting the wireframe assembly on a mandrel incorporating the same bend curvature and dip molding. Several layers of silicone dispersion can be applied to embed the wireframe and provide the desired thickness. A dispersion of elastomeric material may be applied to the internal surfaces of the cannula assembly 200 for fully embedding the wireframe assembly and building up the wall thickness. In one embodiment, the internal surfaces may be cured in air without contact to any mold forms so as to ensure that these surfaces are ultra smooth and limit the adhesion of blood platelets with the vascular prosthesis.

In reference again to the preferred embodiment of FIGS. 11A-15, components are subsequently added to cannula base assembly 201 as part of cannula assembly 200. At the distal end 202, a support collar 220, an adjustable restraint 230 and an elastomeric gasket 240 are provided for effectively securing the device to a hollow vessel, such as a ventricle of the heart, and for ensuring a fluid tight seal at the interface with the vessel wall. The surrounding tissue of the vessel such as the left ventricle of the heart is restrained between intraventricular flange 204 of cannula base assembly 201 and elastomeric gasket 240. The portions of the device that maintain contact with the tissue include the flange shoulder 206, distal straight portion 205 of cannula base assembly 201 and the tissue bearing region 241 of elastomeric gasket 240. One or more of these surfaces are optionally layered with a flocking material, or textured as described above, to promote tissue in-growth.

As best seen in FIG. 12, an elastomeric gasket 240 may be configured as an axially symmetric structure with a central hole for fitting over straight region 205 of base assembly 201. The internal diameter of surface 243 of elastomeric gasket 240 may be sized for an interference fit with the base assembly 201 and to provides a seal with respect to the external elastomeric material of cannula base assembly 201. As shown, the elastomeric gasket 240 may include ribs on a tissue bearing or contacting region 241 to facilitate a seal and provide hemostasis when constrained against the exterior vessel tissue at an anastomosis site without needing to apply excessive compressive force to elastomeric gasket 240. By reducing the compressive force required for fixation and hemostasis, the device is less prone to pull out of the vessel under a tension applied to the conduit. Additionally, tissue necrosis at the vascular interface can be avoided.

A support collar 220 and adjustable restraint 230 enable adjustable positioning of elastomeric gasket 240, including the ability to apply sufficient but not excessive compression against the vessel tissue, such as the myocardium, when affixed to the heart. The actual distance between intraventricular flange 204 and elastomeric gasket 240 will be dependent on the tissue thickness at the anastomosis. In one embodiment, the opposing shoulder 242 of the elastomeric gasket 240 bears against distal flange 232 of adjustable restraint whereas adjustable restraint 240 interlocks with respects to support collar 220 which is attached to cannula base assembly 206.

As seen in FIGS. 12-14, the support collar 220 includes a tubular structure with a grooved region 223 along an outer surface. The support collar additional includes internal grooves 222 and radial holes 224 that help enable the support color 220 to maintain a fixed position when bonded to cannula base assembly 201 such as with an elastomeric adhesive such a silicone. In such a case, the internal grooves 222 and radial holes 224 become filled with adhesive to provide a mechanical interlock as well as an adhesive bond between the elastomeric material and the support collar 220. The material of support collar 220 may include, for example, polycarbonate or some other relatively rigid and biocompatible material that may be primed for a good adhesion with silicone.

The adjustable restraint 230 is best seen in FIG. 15 and includes a body 231 coupled to a distal flange 232 by way of a plurality of slender strut segments 233. Since the attachment of body 231 is interrupted with large cutouts between sets of strut segments 233, the stiffness of body 231 is substantially independent from the stiffness of distal flange 232. This enables the body 231 to be deformed by squeezing tab features 234 that are on opposing sides of the ring shaped structure. At an orientation that is substantially perpendicular to the tabs 234 are two regions of internal locking grooves 235. The body 231 is sufficiently thin to enable flexure so that when the tabs 234 are pushed together (such as with finger pressure), the internal locking grooves 235 are displaced from one another. When the tabs 234 are released, the body 231 assumes its normal relaxed geometry and internal locking grooves 235 return to their normal positions relative to one another.

As best seen in FIG. 12, when the adjustable restraint 230 is attached to the support collar 220, the internal locking grooves 235 of the adjustable restraint 230 matingly engage the grooved region 223 of the support collar 220. When it is desired to move the adjustable restraint 230 along the support collar 220, the tabs 234 of adjustable restraint 230 can be pushed together to temporarily deform the adjustable restraint into an oblong shape such that the internal grooves 235 are displaced away from one another and disengage the grooved regions 223 of the support collar 220. One particular advantage of such an embodiment as compared to an embodiment utilizing a threaded nut or flange exhibiting an interference fit is that is that adjustable restraint can be adjusted without friction or threading so that the surgeon and readily determine the amount of pressure being applied around the vessel wall. Thus, the risk of excessive pressure which might cause the intraventricular flange 204 to pull out from within the vessel is reduced. This is accomplished while still providing sufficient pressure to ensure fixation and hemostasis. Moreover the currently described embodiment enables the relatively quick performance of an anastomosis while rapidly achieving hemostasis with minimal blood loss since the process of attaching the cannula assembly and achievement of hemostasis is time critical. An instantaneous method is especially desirable for performing an anastomosis on a beating heart as may be the case for a minimally invasive procedure in which the device is installed without a thorocotomy or cardiopulmonary bypass.

In reference to FIGS. 11A-11C and 12 components for attaching the cannula assembly 200 to a blood flow device, such as a blood pump, may also be integrated along the distal end 203 of cannula assembly 200. For example, this may include a coupling fitting 250 and a compression ring 260. The portions of the coupling fitting 250 that interface with the blood flow device are a tubular segment 251 and a locking flange 252. The tubular segment 251 facilitates precise concentric alignment of the mating components and also provides a cylindrical surface for an o-ring or other seal that may be housed within a mating blood flow device. The locking flange 252 is shown as an outward projecting feature of the coupling fitting 250 that may be used for securing the cannula assembly 200 to a mating blood flow device, wherein the locking flange 252 is made captive and the cannula assembly 200 is prevented from unintentionally detaching from the mating blood device.

As best seen in the section view of FIG. 12, the coupling fitting 250 may further comprise a barbed segment 253 that fits within proximal end 210 of cannula base assembly 201. In such an embodiment, the elastomeric material of distal portion 210 of cannula base assembly 201 is compressed into barb segment 253 of coupling fitting 250 utilizing a compression ring 260. On account of the relatively undersized diameter of inside surface 261 of the compression ring 260, substantial compression is applied when directed over the proximal end 210. The compression of the elastomeric material and the presence of the wireframe matrix of cannula base assembly 201 from the distal end 205 through the proximal end 210 ensure secure attachment of the fitting 250 when the device is subjected to tension or other loading conditions.

It is noted that other components may also be provided to assist with implant of the cannula assembly 200 or when the cannula assembly is disconnected from an associated blood flow device for any reason (including explant). For example, while not explicitly shown, a plug may be fitted to the proximal end 210 of the cannula base assembly 201. The plug may be used to prevent undue blood flow through the cannula assembly 200 during various procedures when it is not yet connected to all of its associated devices or components. In one embodiment, such a plug may configured to just cap off the proximal end of the cannula assembly 200. In another embodiment, a flexible plug may be configured to substantially fill flow path defined by the cannula assembly 200 between the distal end 205 and the proximal end 210. Such a plug may be removed when it is desired to couple (or re-couple) the cannula assembly 200 with an associated blood flow device.

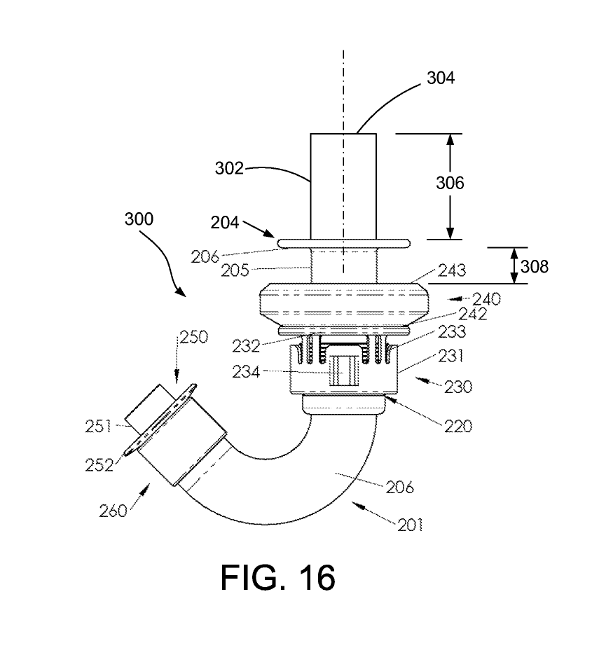

Referring now to FIG. 16, another cannula assembly 300 is shown. The cannula assembly 300 is substantially similar to that which is described above with respect to FIGS. 11A-11C, 12 and 13. The cannula assembly 300, however, includes an additional tubular extension 302 that extends distally beyond the flange 204 (which may include a conformal flange assembly such as has been described hereinabove) and has an open end 304 for fluid communication with a hollow body, such as the ventricle of a heart. In one embodiment, the tubular extension extends a fixed distance 306 beyond the flange 204, while the distance 308 between the flange 204 and the gasket 240 (or other adjustable coupling structure) is variable. Such an embodiment enables the doctor to position the cannula assembly 300 such that the inlet (i.e., open end 304) is positioned a desired depth within a ventricle (or other vessel). The flange 204 acts as a depth gauge or a guide so that the person implanting the assembly 300 does not have make a judgment as to depth, nor is additional imaging required to determine placement of the cannula assembly.

It is noted that by positioning the open end 304 of the tubular extension away from the tissue and at a predetermined distance within the vessel, certain complications may be avoided. for example, such may help to prevent trabeculations from being pulled within the flow path of the cannula assembly. Additionally, such an embodiment helps to prevent endothelization over the inlet of the cannula assembly 300.

In one embodiment, the tubular extension 302 may be formed as a substantially rigid structure. In another embodiment, the tubular extension 302 may be flexible and may be formed similar to various tubular portions of cannula assembly 300 described hereinabove. Additionally, while shown as extending substantially straight along a longitudinal axis, the extension may be formed to exhibit a desired bend so as to position the open end 304 at a desired location within a particular vessel. Further, while the open end is shown to be substantially normal to the longitudinal axis, it may be formed at an angle or exhibit a curve if desired to provide particular flow characteristics within a vessel. It is also noted that such a tubular extension may be incorporated into any of the described embodiments set forth herein.

Various advantages are provided by the present invention. Some non-limiting examples of advantages and benefits include: optimal positioning on a ventricular apex to eliminate the occurrence of inlet impinging on an opposing wall (e.g., septal wall) and substantially restricting inflow during systole; an improved cannula inlet with a larger mouth than conventional systems; improved hemodynamic characteristics of the system resulting in less trauma to the blood, reduced thrombogenicity, and reduced hemodynamic impedance of the VAD system; mobility of the myocardium so as to maintain conformity during changes in curvature; a structure that effectively seals the ventricle at the cannula joint and enables homeostasis; a structure that adjustably secures the cannula inlet and is, thus, adaptable to various myocardial thicknesses; a structure to attach the cannula to the ventricle without sutures if desired; a reduction in the time needed to perform the associated medical procedures; the composite structure of a flange assembly enables a retention flange of sufficient stiffness, yet of minimal thickness, so as to efficiently collapse within a small cross-section. Of course other benefits and advantages will be recognized by those of ordinary skill in the art.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention includes all modifications, equivalents, and alternatives falling within the spirit and scope of the invention as defined by the following appended claims.

What is claimed is:

1. A cannula assembly comprising:
a tubular structure coupled with a flange assembly, wherein the flange assembly includes a plurality of wireform loops disposed in a circumferential, woven pattern about an end of the tubular structure, the flange assembly being configured to exhibit a first, collapsed state wherein the plurality of wireform loops extend substantially axially from the tubular structure, and a second, expanded state wherein the plurality of wireform loops extend in a direction having a substantial radial component relative to the tubular structure.

2. The assembly of claim 1, wherein the plurality of wireform loops are mechanically coupled to the tubular structure.

3. The assembly of claim 1, wherein the circumferential, woven pattern of the plurality of wireform loops defines a plurality of wire crossings and a plurality of internal openings.

4. The assembly of claim 3, wherein the flange assembly further includes an enclosure material for covering the plurality of internal openings defined by the plurality of wireforms.

5. The assembly of claim 4, wherein the enclosure material seamlessly extends continuously through an interior of the tubular structure.

6. The assembly of claim 4, wherein the enclosure material is bonded to the wireform loops and provides a webbing within the plurality of openings defined by the plurality of wireform loops.

7. The assembly of claim 4, wherein the enclosure material includes a segmented polyurethane.

8. The assembly of claim 4, wherein the enclosure material includes silicone.

9. The assembly of claim 1, wherein the flange assembly is of sufficient flexibility to conform to a varied anatomical geometry.

10. The assembly of claim 1, wherein the connector is sized and configured to fluidly connect with a chamber of the human heart.

11. The assembly of claim 1, wherein the flange assembly includes an odd number of wireforms.

12. The assembly of claim 11, wherein the number of wireform loops is within an inclusive range of 5 to 11.

13. The assembly of claim 1, wherein each of the wireform loops includes a first terminal end and a second terminal end, the first and second terminal ends being positioned approximately 180° apart from each other on a circumferential periphery of the tubular structure.

14. The assembly of claim 1, wherein an axial thickness of a radial flange portion of the flange assembly is approximately twice a cross-sectional thickness of a wireform loop of the plurality of wireform loops.

15. The assembly of claim 1, wherein an outer diameter of the flange assembly is more than approximately twice a diameter of the tubular portion when the flange assembly is in the expanded state.

16. The assembly of claim 1, further comprising a sleeve at least partially disposed within the tubular structure.

17. The assembly of claim 1, wherein the tubular structure is substantially impermeable to air.

18. The assembly of claim 1, wherein the tubular structure includes an exterior surface formed of material that promotes tissue in-growth.

19. The assembly of claim 1, wherein a proximal surface of the flange assembly includes an exterior surface formed of material that promotes tissue in-growth.

20. The assembly of claim 1, wherein the connector further comprises a sewing ring positioned about the tubular structure at a position proximal to the flange assembly.

21. The assembly of claim 1, further comprising an adjustable flange member associated with the tubular structure proximal of the flange assembly.

22. The assembly of claim 1, wherein the at least some of the plurality of wireforms extend from the flange assembly and define, at least in part, the tubular structure.

23. The assembly of claim 22, wherein the wireforms that define the tubular structure are woven along at least a portion of the length of the tubular structure.

24. The assembly of claim 22, wherein the wireforms that define the tubular structure transition between a woven configuration and a helical configuration.

25. The assembly of claim 22, wherein the wireforms that define the tubular structure transition from a first woven section to a helical coil and from the helical coil to a second woven section.

26. The assembly of claim 1, wherein the tubular portion is flexible and can bent at an angle without kinking

27. The assembly of claim 1, wherein the tubular portion is configured to exhibit a bend at an acute angle without any substantial stress.

28. The assembly of claim 1, wherein the tubular structure extends distally beyond the flange assembly when the flange assembly is in the expanded state.

29. The assembly of claim 28, wherein a distance between a distal end of the tubular structure and a location wherein the flange assembly is coupled with the tubular structure is fixed.

30. A cannula assembly comprising:
a tubular structure coupled with a conformal flange, wherein the conformal flange assembly is configured to exhibit a first, collapsed state and a second, expanded state wherein the conformal flange extends substantially radially outward relative to the tubular structure, wherein a proximal surface of the conformal flange is formed of a material that promotes tissue in-growth.

31. A method of coupling a cannula to a tissue structure, the method comprising:
collapsing a flange assembly within a delivery member;
passing the delivery member through an opening in the tissue structure;
expanding the flange assembly to exhibit a size greater than the opening in the tissue structure;
conforming the flange assembly to the anatomy of the tissue structure;
promoting tissue in-growth between the tissue structure and the flange assembly.

32. The method of claim 31, further comprising forming the flange assembly of a plurality of woven wireform loops.

33. The method of claim 31, further comprising providing a tubular structure associated with the flange assembly, and extending a free end of the tubular structure distally beyond the flange assembly a defined distance, and extending the tubular structure proximally of the flange assembly for connection with another structure.

34. A cannula assembly comprising:
a tubular structure coupled with a flexible, conformal flange, wherein the conformal flange assembly is configured to exhibit a first, collapsed state and a second, expanded state wherein the conformal flange extends substantially radially outward relative to the tubular structure, and wherein the tubular structure extends both distally and proximally of the conformal flange.

35. The assembly of claim 34, further comprising a connection structure adjustably positioned about the tubular structure proximal of the conformal flange.

36. The assembly of claim 35, further comprising an inlet in the tubular structure distal of the flange, wherein the flange is fixed relative to its distance along the tubular structure to the inlet.
